(19) United States
(12) Patent Application Publication
(10) Pub. No.: US 2019/0262064 A1
(43) Pub. Date: Aug. 29, 2019

(54) MEDICAL APPARATUS

(71) Applicant: OLYMPUS CORPORATION, Tokyo (JP)

(72) Inventor: Tsunetaka AKAGANE, Hachioji-shi (JP)

(73) Assignee: OLYMPUS CORPORATION, Tokyo (JP)

(21) Appl. No.: 16/407,287

(22) Filed: May 9, 2019

Related U.S. Application Data
(63) Continuation of application No. PCT/JP2016/083225, filed on Nov. 9, 2016.

(51) Int. Cl. A61B 18/14

(52) U.S. Cl. CPC A61B 18/1445; A61B 2017/320094

(57) ABSTRACT

A medical apparatus includes a rod, a heat-insulating coat having a heat insulating property, an electrically conductive coat having an electric conductive property. The heat-insulating coat is formed on at least a part of the rod in a circumferential direction of the rod. The heat-insulating coat is configured to suppress conduction of heat from a treatment target to the rod. The electrically conductive coat is formed on the heat-insulating coat and contacts the treatment target. The electrically conductive coat is configured to treat the treatment target using high-frequency energy.

### CROSS-REFERENCE TO RELATED APPLICATIONS

This application is a Continuation Application of PCT Application No. PCT/JP2016/083225, filed Nov. 9, 2016, the entire contents of which are incorporated herein by reference.

### BACKGROUND

The present disclosure relates to a medical apparatus.

There is known a treatment instrument that performs a treatment such as hemostasis or cutting, by applying heat energy to, and denaturing, a biological tissue such as a blood vessel. This kind of medical apparatus can include a heater unit including a heat generating element. The heater unit is configured to raise the temperature of the biological tissue up to such a temperature that the biological tissue can be treated. The heat of the heater unit, the temperature of which is sufficiently raised, is applied to the biological tissue, and the biological tissue is treated.

For example, as disclosed in Jpn. Pat. Appln. KOKAI Publication No. 2006-305236, there is known a treatment instrument in which a coating portion formed of a non-adhesive coating material is provided on the surface of the heater unit.

### SUMMARY

A medical apparatus can include a rod, a heat-insulating coat having a heat insulating property, an electrically conductive coat having an electric conductive property. The heat-insulating coat is formed on at least a part of the rod in a circumferential direction of the rod. The heat-insulating coat is configured to suppress conduction of heat from a treatment target to the rod. The electrically conductive coat is formed on the heat-insulating coat and at a position to be in contact with the treatment target. The electrically conductive coat is configured to treat the treatment target by high-frequency energy.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view illustrating a medical system including a medical apparatus.

FIG. 2 is a side view illustrating an end effector of the medical apparatus.

FIG. 3A is a cross-sectional view illustrating the end effector taken along the line IIIA-IIIA in FIG. 2.

FIG. 3B is a cross-sectional view illustrating a treatment section of the end effector taken along the line IIIB-IIIB in FIG. 3A.

FIG. 4 is a cross-sectional view illustrating an exemplary embodiment.

FIG. 5 is a cross-sectional view illustrating an exemplary embodiment.

FIG. 6 is a cross-sectional view illustrating an exemplary embodiment.

FIG. 7 is a schematic view illustrating a medical system including a medical apparatus with a medical instrument.

FIG. 8 is a cross-sectional view illustrating a transducer of the medical apparatus.

FIG. 9 is a side view illustrating an end effector of the medical apparatus of a medical instrument.

FIG. 10 is a cross-sectional view illustrating the end effector taken along the line X-X in FIG. 9.

FIG. 11 is a cross-sectional view illustrating a treatment section of the end effector of the medical instrument of an exemplary embodiment.

FIG. 12 is a cross-sectional view illustrating a modification of an exemplary embodiment.

FIG. 13 is a cross-sectional view illustrating a modification of an exemplary embodiment.

### DETAILED DESCRIPTION OF THE INVENTION

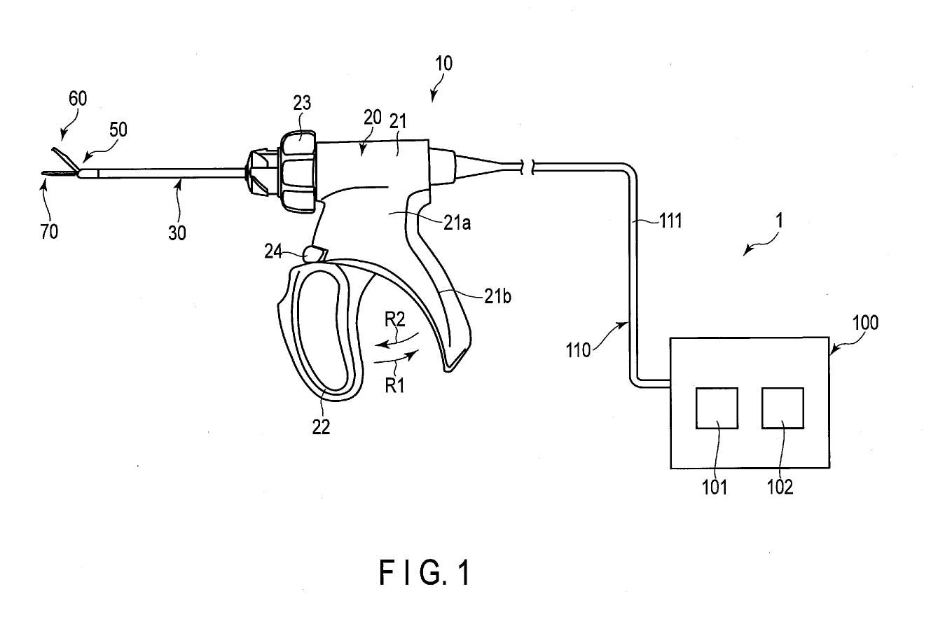

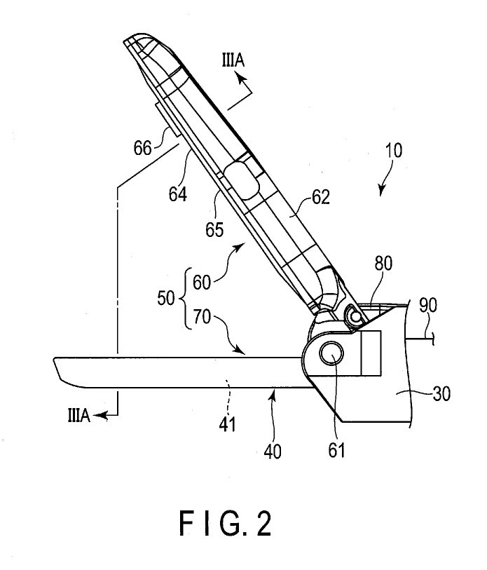

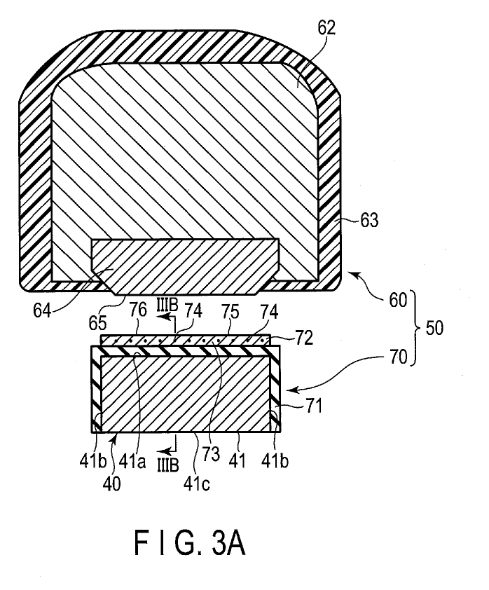

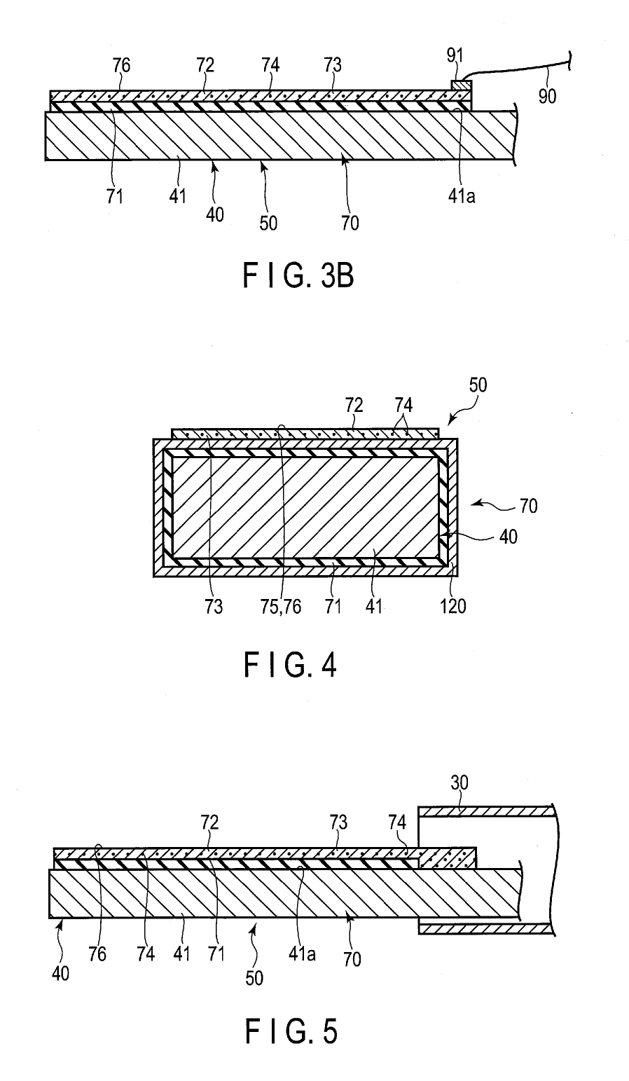

A medical system (treatment system) 1 including a medical apparatus (medical instrument/treatment instrument) 10 will now be described with reference to FIG. 1 to FIG. 6. FIG. 1 is a schematic view illustrating a medical system 1 including the medical apparatus 10. FIG. 2 is a side view illustrating an end effector 50 of the medical apparatus 10. FIG. 3A is a cross-sectional view illustrating the end effector 50. FIG. 3B is a cross-sectional view illustrating a rod-side treatment section 70 of the end effector 50.

As illustrated in FIG. 1, the medical system 1 includes the medical apparatus 10 which can treat a treatment target; an energy control device (electric power source) 100 which can supply energy to the medical apparatus 10; and a cable 110 which is connected to the medical apparatus 10 and energy control device 100 and can transmit energy from the energy control device 100 to the medical apparatus 10.

In the present embodiment, the medical apparatus 10 includes, for example, a bipolar-type treatment instrument such as an electric scalpel. A target of treatment by the medical apparatus 10 is, for example, a biological tissue, a blood vessel, etc. The medical apparatus 10 is an example of the medical instrument of the present invention.

The medical apparatus 10 includes a hand piece 20 including an operation section configured to be operated by a surgeon; a shaft 30 coupled to the hand piece 20 and partly disposed within the hand piece 20; a rod 40 disposed within the shaft 30 and hand piece 20; an end effector 50 configured to clamp a treatment target by opening and closing, and configured to perform treatment on the treatment target; and a wiring line 90 (shown in FIG. 3B) configured to transmit energy to the end effector 50.

The hand piece 20 is formed in such a shape that the surgeon can grasp the hand piece 20. The hand piece 20 includes a housing 21 which forms an exterior of the hand piece 20; a handle 22 which is an example of an operation section provided on the housing 21; a rotation knob 23 provided on the housing 21 such that the rotation knob 23 is rotatable around an axis of the housing 21; and an operation button 24 which is an example of the operation section provided on the housing 21.

When the rotation knob 23 is rotated relative to the housing 21 by a publicly known mechanism, the shaft 30, the rod 40 and a jaw 60 (to be described later) rotate together with the rotation knob 23 relative to the housing 21.

The housing 21 includes a housing body 21a provided on the rotation knob 23; and a grip 21b formed such that the surgeon can grasp the grip 21b. The housing body 21a is a portion disposed along the axis of the shaft 30 and an extension line of the axis in the housing 21.

The handle 22 is disposed in a position opposed to the grip 21b. One end of the handle 22 is supported by the housing body 21a so as to be movable relative to the grip 21b. The handle 22 is swingable in a direction R1 in which the other end portion of the handle 22 moves toward the grip 21b, and in a direction R2 in which the distal portion moves away from the grip 21b.

In the present embodiment, an operation of swinging the handle 22 in the direction R1 in which the other end portion of the handle 22 moves toward the grip 21b is defined as an operation of closing the handle 22. An operation of swinging the handle 22 in the direction R2 in which the other end portion of the handle 22 moves away from the grip 21b is defined as an operation of opening the handle 22.

The operation button 24 is disposed on the housing body 21a in the vicinity of the handle 22. The surgeon can operate the operation button 24 at the same time as operating the handle 22. An operation on the operation button 24 is, for example, an operation of pushing the operation button 24.

The operation button 24 is an example of the operation section which is operated to supply energy from the energy control device 100 to the medical apparatus 10. In the medical system 1, when the operation button 24 is operated by the surgeon, energy is supplied from the energy control device 100 to the medical apparatus 10.

The shaft 30 is formed, for example, in a cylindrical shape. The shaft 30 has such rigidity as to be capable of withstanding a load which is input due to, for example, a contact with a treatment target, when the treatment target is treated. The shaft 30 is formed of a metallic material. The shaft 30 may partly be formed of a material with electrical conductivity. Here, one end of the shaft 30, which is disposed outside the housing 21, is defined as a distal end, and the other end of the shaft 30, which is disposed inside the housing 21, is defined as a proximal end.

In the present embodiment, the shaft 30 includes, in the inside thereof, a driving section (driving pipe) 80 (shown in FIG. 2) configured to open and close the end effector 50. The rod 40 is disposed in the inside of the driving section 80 of the shaft 30. The driving section 80 is movable along the axis of the shaft 30.

In this embodiment, a description is given of an example in which the driving section 80 is provided in the inside of the shaft 30 and is movable relative to the outside of the shaft 30 along the axis of the shaft 30. The driving section 80 is electrically connected to a jaw-side electrode 64 (to be described later). Besides, it is also preferable that the driving section 80 is provided on the outside of the shaft 30. In this case, it is preferable that the driving section 80 is provided on the outside of the shaft 30 and is movable relative to the inside of the shaft 30 along the axis of the shaft 30. The shaft 30 is electrically connected to the jaw-side electrode 64 (to be described later).

In the present embodiment, the driving section 80 moves toward the distal side of the shaft 30 in accordance with the operation of closing the handle 22 toward the grip 21b of the housing 21. The driving section 80 moves toward the proximal side of the shaft 30 in accordance with the operation of opening the handle 22 away from the grip 21b of the housing 21. A driving force by the operation of the handle 22 is transmitted to the driving section 80 by a publicly known mechanism in the shaft 30 and housing 21.

As illustrated in FIG. 1, the rod 40 is formed in a rod shape and is disposed in the shaft 30. The rod 40 is formed of a metallic material which is an example of a material having a proper rigidity. It should suffice if the rod 40 has enough strength to perform treatment, and the material of the rod 40 is not limited to the metallic material. The rod 40 has such strength as to be capable of treating a treatment target. It is also preferable that the rod 40 is formed of, for example, a material having electrical conductivity. That is, it is preferable that the rod 40 has an electric conductive property.

One end portion of the rod 40 on the distal side of the shaft 30 and a portion (distal portion) near the one end position are disposed outside from the opening of the distal end of the shaft 30. Here, that portion of the rod 40, which is located outside the shaft 30, is defined as a distal portion 41.

Although not illustrated, the other end portion (proximal portion) of the rod 40 on the proximal side of the shaft 30 is disposed within the housing 21. Note that the other end of the rod 40 may be disposed, for example, within the shaft 30, or may be disposed out of the proximal end of the shaft 30.

The rod 40 is supported and fixed by the shaft 30. The fixation of the rod 40 to the shaft 30 is made by, for example, an annular insulating member (rubber lining) disposed in the shaft 30. An outer peripheral surface of the insulating member is fixed to an inner peripheral surface of the shaft 30. The rod 40 is disposed in the inside of the insulating member, and an inner peripheral surface of the insulating member is fixed to the rod 40.

Thus, the rod 40 is not electrically connected to the shaft 30. The rod 40 rotates together with the shaft 30 around the center axis of the rotation knob 23 by publicly known art. The movement of the rod 40 relative to the shaft 30 in the axial direction of the center axis of the rotation knob 23 is prevented.

As illustrated in FIG. 2, the end effector 50 includes a jaw (jaw assembly) 60 rotatably supported on a distal portion of the shaft 30, and a rod-side treatment section 70 provided on the distal portion 41 of the rod 40.

The jaw 60 is an example of a treatment section which is able to perform treatment on the treatment target in cooperation with the rod-side treatment section 70. The jaw 60 is configured to clamp the treatment target between the jaw 60 and an electrically conductive coat 72. The jaw 60 is rotatably coupled to the distal portion of the shaft 30 by a support pin 61.

The support pin 61 is provided on the shaft 30 in such a position that the axis of the support pin 61 is perpendicular to the axis of the shaft 30 (the center axis of the rotation knob 23). By the opening/closing operation of the handle 22 relative to the housing 21, the jaw 60 can rotate around the support pin 61 between an opposed position where the jaw 60 is opposed to the rod 40 and a separated position where the jaw 60 is spaced apart from the rod 40 as illustrated in FIG. 2.

FIG. 3A illustrates a state in which the end effector 50 is cut along a cross section perpendicular to the axis of the distal portion 41 of the rod 40, and cut along a cross section perpendicular to the axis of the jaw 60. As illustrated in FIG. 3A, the jaw 60 includes a jaw body 62 coupled to the shaft 30 by the support pin 61; a cover 63 covering the outside of the jaw body 62; and a jaw-side electrode 64. The jaw-side electrode 64 is supported on the jaw body 62 at a position opposed to the distal portion 41 of the rod 40. The jaw-side electrode 64 is electrically connected to, for example, the driving section 80 of the shaft 30 by publicly known art. The jaw-side electrode 64 is configured to pass the high-frequency energy between the jaw-side electrode 64 and the electrically conductive coat 72.

The jaw 60 is disposed such that the longitudinal direction of the jaw 60 extends along an extension line of the axis of the shaft 30 when the jaw is closed against the distal portion 41. A proximal portion of the jaw body 62 is coupled to the shaft 30 by the support pin 61. The cover 63 is formed of an electrically insulating material such as a resin material.

In the jaw-side electrode 64, a counter-surface 65, which is opposed to the distal portion 41 of the rod 40, can be formed in a proper shape. Here, for the purpose of simple description, the counter-surface 65 is formed, for example, in a planar shape. As illustrated in FIG. 2, a spacer 66 with an electrical insulation property is provided on the counter-surface 65 of the jaw-side electrode 64. The spacer 66 prevents short-circuit by coming in contact with an electrically conductive coat 72 (to be described later).

The rod-side treatment section 70 includes the distal portion (base portion) 41 of the rod 40, a heat-insulating coat (heat-insulating layer) 71 formed on the distal portion 41 of the rod 40, and an electrically conductive coat (electrically conductive layer) 72 formed on the heat-insulating coat 71.

The distal portion 41 of the rod 40 is formed of a rigid member as an example. The rigid member has such strength as to be capable of treating the treatment target. It is assumed that the distal portion 41 of the rod 40 is formed to have, for example, a rectangular cross section perpendicular to the axis of the rod 40. It is assumed that a surface 41a of the distal portion 41 on the jaw 60 side is formed as a planar surface. The surface 41a of the distal portion 41 is formed as a planar surface which is parallel or substantially parallel to the counter-surface 65 of the jaw body 62 of the jaw 60 when the jaw 60 is closed against the distal portion 41.

The heat-insulating coat 71 has a heat insulating property. The heat-insulating coat 71 is formed at least on a position in the distal portion 41, which is opposed to the counter-surface 65 of the jaw-side electrode 64. The heat-insulating coat 71 is formed on the surface 41a of the distal portion 41 of the rod 40, and on both side surfaces 41b between which the surface 41a is interposed. The heat-insulating coat 71 is configured to suppress conduction of heat from a treatment target to the rod 40. As illustrated in FIG. 3A, in the present embodiment, the heat-insulating coat 71 is not formed on a surface 41c opposite to the surface 41a.

The heat-insulating coat 71 is configured to make it difficult for the heat of the electrically conductive coat 72 to be conducted to the rod 40. The specific heat capacity of the heat-insulating coat 71 is greater than the specific heat capacity of the rod 40. The thermal conductivity of the heat-insulating coat 71 is lower than the thermal conductivity of the rod 40.

The heat-insulating coat 71 is formed of a material having a greater specific heat capacity than the specific heat capacity of the rod 40 and having a lower thermal conductivity than the thermal conductivity of the rod 40. The material, of which the heat-insulating coat 71 is formed, is, for example, a ceramic material or a heat-resistant material.

In the present embodiment, the heat-insulating coat 71 is formed to have a thickness in a range of, e.g. several μm to several-hundred μm. The heat-insulating coat 71 has a porous structure as a whole. The heat-insulating coat 71 is formed of, for example, a material in which particles with heat-insulating properties are dispersedly mixed in a base material that is formed of PEEK resin or the like.

The material used for the base material in the heat-insulating coat 71 may be a material other than PEEK. The particles are formed of hollow, spherical glass (soda-lime borosilicate glass) or silica (silicon dioxide), but may be formed of other materials.

For example, a space filled with air is provided in the inside of the particle. Thus, the particle can exhibit adiathermancy. The diameters of the particles are not uniform, and particles of various grain sizes are mixedly present. In any case, the diameter of each particle is less than the thickness of the heat-insulating coat 71. The shape of the particle is not limited to the spherical shape, and may be other various shapes such as an ellipsoidal shape or a thin scale-like shape.

The heat-insulating coat 71 electrically insulates the electrically conductive coat 72 from the rod 40. The electrically conductive coat 72 has an electric conductive property. The electrically conductive coat 72 is prevented from coming in contact with the rod 40 by the heat-insulating coat 71.

A heat capacity of the electrically conductive coat 72 is less than that of the rod 40. A thermal conductivity of the electrically conductive coat 72 is lower than that of the rod 40.

The electrically conductive coat 72 is formed on the heat-insulating coat 71 and at a position to be in contact with the treatment target. That is, the electrically conductive coat 72 is opposed to the jaw-side electrode 64. The electrically conductive coat 72 is configured to treat the treatment target by high-frequency energy. Preferably, the electrically conductive coat 72 is formed within the range of the heat-insulating coat 71. The phrase “formed within the range of the heat-insulating coat 71” in this context means that when the electrically conductive coat 72 is directly formed on the heat-insulating coat 71, the electrically conductive coat 72 is disposed inside the peripheral edges of the heat-insulating coat 71. Also when another layer is provided between the electrically conductive coat 72 and heat-insulating coat 71, the electrically conductive coat 72 is disposed inside the peripheral edges of the heat-insulating coat 71.

The position and shape of the electrically conductive coat 72 are determined by the medical apparatus 10. The electrically conductive coat 72 is configured to clamp a treatment target between the electrically conductive coat 72 and jaw-side electrode 64 and configured to apply high-frequency energy (high-frequency current) with the jaw-side electrode 64 to the treatment target. The electrically conductive coat 72 is used as an electrode configured to pass the high-frequency energy through the treatment target clamped between the electrically conductive coat 72 and the jaw-side electrode 64.

Thus, the range of formation of the heat-insulating coat 71 is determined by the position of the electrically conductive coat 72 and the shape of the electrically conductive coat 72. In other words, the heat-insulating coat 71 is formed such that the electrically conductive coat 72, which is determined as described above, can be disposed in the range of the heat-insulating coat 71. Note that the positional relationship between the electrically conductive coat 72 and heat-insulating coat 71, and the factors that determine the positional relationship, are not limited to those described above.

In the present embodiment, the electrically conductive coat 72 is formed on that part of the heat-insulating coat 71, which is formed on the surface 41a of the distal portion 41 of the rod 40. The electrically conductive coat 72 has such a size as to fall within the inside of the peripheral edges of that part of the heat-insulating coat 71, which is formed on the surface 41a. In this embodiment, the electrically conductive coat 72 is disposed in the inside of the heat-insulating coat 71, and is not directly connected to the rod 40.

The electrically conductive coat 72 is opposed to the counter-surface 65 of the jaw-side electrode 64. A surface 75 of the electrically conductive coat 72 serves as a treatment surface 76 which comes in contact with a biological tissue. The electrically conductive coat 72 has an area and a shape configured to properly treat the biological tissue.

The electrically conductive coat 72 has electrical conductivity. The electrically conductive coat 72 is formed of a base material (electrically non-conductive coat) 73 and a plurality of (many) particles (electrically conductive particles) 74.

The base material 73 is formed of a material with proper heat resistance, electrical insulation properties and water repellency. The base material 73 is formed of, for example, a material in which a fluororesin such as PTFE and a binder such as a polyamide-imide are mixed. In the present embodiment, the electrically conductive coat 72 has a less specific heat capacity and a lower thermal conductivity than the rod 40. Thus, the electrically conductive coat 72 has a higher heat radiation property than the distal portion 41. In other words, the electrically conductive coat 72 can be cooled more easily than the distal portion 41.

The particles 74 are formed of an electrically conductive material. Particles with electrical conductivity are, for example, metallic materials. In the present embodiment, the particles are, for example, silver particles. The particles are formed in various shapes. The shapes include, for example, a spherical shape, and a scale-like shape. The particles may include two or more kinds of shapes. The particles 74 are dispersed and distributed in the base material 73. A large number of (countless) particles 74 are disposed in the base material 73 and form many electrical conduction paths, so that the electrically conductive coat 72 may have electrical conductivity as a whole.

Accordingly, the electrically conductive coat 72 has electrical conductivity. The electrically conductive coat 72 receives heat from the treatment target through which electric energy was passed and the temperature of which was raised. The heat is conducted from the electrically conductive coat 72 to the heat-insulating coat 71. The heat-insulating coat 71 has a greater specific heat capacity and a lower thermal conductivity than the rod 40.

When the heat generated in the electrically conductive coat 72 is conducted to the distal portion 41 of the rod 41 through the heat-insulating coat 71, the heat conducted to the heat-insulating coat 71 from the electrically conductive coat 72 is conducted to the rod 40 side along paths detouring the particles in the heat-insulating coat 71.

Thus, in the heat-insulating coat 71, by the presence of many (countless) particles, the distance of heat conduction in the thickness direction of the heat-insulating coat 71 becomes longer than the actual thickness of the heat-insulating coat 71. Therefore, in the heat-insulating coat 71, since the heat flux (heat transfer amount per unit time) in a direction penetrating the heat-insulating coat 71 decreases, the conduction of heat from the electrically conductive coat 72 to the rod 40 is prevented.

The end effector 50 can clamp the treatment target between the jaw 60 and the rod-side treatment section 70, by the opening/closing of the jaw 60 relative to the rod-side treatment section 70.

In the present embodiment, the rod 40 and wiring line 90 are electrically insulated. As illustrated in FIG. 3B, the wiring line 90 is disposed between the rod 40 and driving section 80, or between the driving section 80 and shaft 30. One end of the wiring line 90 is electrically connected to the electrically conductive coat 72.

FIG. 3B illustrates a state in which the rod-side treatment section 70 is cut along a cross section which is perpendicular to the width direction of the distal portion 41 and extends along the axis of the distal portion 41. The wiring line 90 is connected to the electrically conductive coat 72 by a connection portion 91. The connection portion 91 is formed of, for example, an electrically conductive material having the same composition as the electrically conductive coat 72.

A description is now given of an example of the method of fixing the connection portion 91 to the electrically conductive coat 72. To begin with, an electrically conductive material, of which the electrically conductive coat 72 is formed, is applied to the heat-insulating coat 71. The electrically conductive material, of which the electrically conductive coat 72 is formed, includes an organic component and is in a liquid phase. A material, of which the connection portion 91 is formed, is applied to the wiring line 90. In the present embodiment, the connection portion 91 is formed of the same material as the electrically conductive coat 72.

Next, a heat treatment is performed to provisionally harden (provisionally dry) the materials which were applied to the heat-insulating coat 71 and wiring line 90 and of which the electrically conductive coat 72 is formed. The heat treatment is performed by leaving the materials in a furnace set at, e.g. 60° C. to 120° C. for a predetermined time.

After the end of the provisional hardening, the heat-treated material, which is applied to the wiring line 90 and of which the electrically conductive coat 72 is formed, is fixed to the heat-treated material, which is applied to the heat-insulating coat 71 and of which the electrically conductive coat 72 is formed. Next, a heat treatment is performed to properly harden (bake) these materials.

The heat treatment for proper hardening is performed by leaving the materials, for a predetermined time, in the furnace set at, e.g. 200° C. to 330° C., which are higher than the temperatures in the heat treatment for the provisional hardening. By the proper hardening, the electrically conductive coat 72 and connection portion 91 are formed, and the electrically conductive coat 72 and connection portion 91 are fixed. Since the connection portion 91 is fixed to the electrically conductive coat 72, the wiring line 90 is electrically connected to the electrically conductive coat 72 via the connection portion 91.

In the state in which the electrically conductive coat 72 and connection portion 91 are properly hardened (baked), the organic component (liquid component) included in the base material is vaporized, and the electrically conductive particles included in the base material are put in contact with each other. Thus, the electrically conductive particles are electrically connected. By the particles being electrically connected, the electrically conductive coat 72 and connection portion 91 have electrical conductivity.

As illustrated in FIG. 1, the energy control device 100 includes a high-frequency energy supply (high-frequency current supply) 101 and a controller 102 configured to control the high-frequency energy supply 101. When the operation button 24 is operated, the controller 102 can supply high-frequency energy from the high-frequency energy supply 101 via the cable 110 to the treatment target that is put in contact with the electrically conductive coat 72 and jaw-side electrode 64 of the medical apparatus 10.

The cable 110 includes a sheath 111 and an electric wire disposed in the sheath 111. The electric wire is electrically connected to the driving section 80 of the shaft 30. The wiring line 90 extends to the cable 110 side through the inside of the shaft 30, and is electrically connected to the electric wire of the cable 110.

Next, the function of the medical apparatus 10 will be described.

When the surgeon treats a treatment target with the medical apparatus 10, the surgeon operates the handle 22. The end effector 50 clamps the treatment target between the jaw-side electrode 64 and electrically conductive coat (electrode) 72. In the state in which the treatment target is clamped by the end effector 50, the surgeon operates the operation button 24, high-frequency energy is applied to the treatment target clamped between the jaw-side electrode 64 and electrically conductive coat (electrode) 72.

When the high-frequency energy is applied to the treatment target, heat is generated in the treatment target by resistance heat generation, and the treatment target is denatured by the heat. By utilizing the denaturing, a treatment such as coagulation, or coagulation/cutting is performed.

The temperature of the electrically conductive coat 72 is raised by the heat generated by the electrical resistance of the electrically conductive coat 72, and by the heat transferred from the treatment target. However, the electrically conductive coat 72 is formed on only that surface of the heat-insulating coat 71, which is opposed to the counter-surface 65 of the jaw 60 in the opening/closing direction of the jaw 60. The heat-insulating coat 71, which makes difficult the transfer of heat, is formed between the electrically conductive coat 72 and the rod 40. The heat-insulating coat 71 has a greater specific heat capacity and a lower thermal conductivity than the rod 40. Thus, the amount of heat, which is transferred from the electrically conductive coat 72 to the rod 40 through the heat-insulating coat 71, is decreased. Heat is not easily conducted from the electrically conductive coat 72 to the heat-insulating coat 71.

The electrically conductive coat 72 has a less specific heat capacity and a lower thermal conductivity than the rod 40. Thus, the electrically conductive coat 72 has a higher heat radiation property than the distal portion 41 and is easily cooled. Thus, heat is prevented from being stored in the electrically conductive coat 72. Therefore, heat is not easily conducted from the electrically conductive coat 72 to the distal portion 41.

In the medical apparatus 10 having the above-described structure, since the heat of the electrically conductive coat 72 is not easily conducted to the distal portion 41, the temperature of the distal portion 41 of the rod 40 can be prevented from rising to a high temperature.

Here, in the rod 40, the distal portion 41, which is a part disposed on the outside of the shaft 30 and constitutes a part of the rod-side treatment section 70, is located close to the treatment target at the time of treatment by the medical apparatus 10, and there is a possibility that the distal portion 41 comes in contact with the vicinity of the treatment target.

However, in the present embodiment, the temperature of the distal portion 41 of the rod 40 can be prevented from rising to a high temperature. Therefore, even if a part other than the treatment surface of the rod-side treatment section 70 comes in contact with the treatment target or the vicinity of the treatment target, high-temperature heat is not conducted to the treatment target, and therefore the treatment target is prevented from being invaded by the heat.

In this manner, according to the medical apparatus 10 of the present embodiment, even if a part other than the treatment surface that is put in contact with the treatment target comes in contact with the treatment target, the treatment target is prevented from being invaded by the contact. Therefore, the safety of the medical apparatus 10, which uses energy for treatment, can be improved.

In the present embodiment, the heat-insulating coat 71 is formed on the surface 41a located on the jaw 60 side in the opening/closing direction, and both side surfaces 41b between which the surface 41a is interposed, in the distal portion 41 of the rod 40 formed in the rectangular cross-sectional shape. The heat-insulating coat 71 is formed on at least a part of the distal portion 41 of the rod 40 in a circumferential direction of the peripheral surface of the distal portion 41 of the rod 40.

The formation of the heat-insulating coat 71 is not limited to the case in which the heat-insulating coat 71 is formed on a part in the circumferential direction of the peripheral surface of the distal portion 41 of the rod 40. As illustrated in FIG. 4, the heat-insulating coat 71 may be annularly formed on the peripheral surface of the distal portion 41 of the rod 40. The heat-insulating coat 71 may be formed on the surface 41a, side surfaces 41b and surface 41c. The heat-insulating coat 71 may be formed on a distal surface of the distal portion 41.

Besides, in the present embodiment, the electrically conductive coat 72 is formed on that surface of the heat-insulating coat 71, which is opposed to the counter-surface 65 of the jaw 60 in the opening/closing direction of the jaw 60. However, the formation of the electrically conductive coat 72 is not limited to the case in which the electrically conductive coat 72 is formed on this surface of the heat-insulating coat 71.

Another layer may be included between the electrically conductive coat 72 and the heat-insulating coat 71. In short, it should suffice if the heat-insulating coat 71 is provided between the electrically conductive coat 72 and the rod 40 so as to make difficult the conduction of heat of the electrically conductive coat 72 to the distal portion 41 of the rod 40. In other words, it should suffice if the electrically conductive coat 72 is provided in an outer layer with respect to the heat-insulating coat 71.

FIG. 4 is a cross-sectional view illustrating an exemplary embodiment. As illustrated in FIG. 4, a reinforcement coat 120 may be formed on the heat-insulating coat 71. The electrically conductive coat 72 may be formed on the reinforcement coat 120. The reinforcement coat 120 has wear resistance. The reinforcement coat 120 is formed such that the reinforcement coat 120 can improve the strength of the rod-side treatment section 70. In this case, too, it is preferable that the electrically conductive coat 72 is formed within the range of the heat-insulating coat 71.

In the present embodiment, the example was described in which the wiring line 90 is used for supplying electric energy (current) to the electrically conductive coat 72. However, the embodiment is not limited to this. For example, as in a modification illustrated in FIG. 5, electric energy may be supplied to the electrically conductive coat 72 by the rod 40.

High-frequency energy is supplied to the rod 40 from the high-frequency energy supply 101 via the cable 110. A part of the electrically conductive coat 72 is electrically connected to the rod 40. As this electrical connection, as illustrated in FIG. 5, the electrically conductive coat 72 may be directly connected to the rod 40.

By the electrical connection of the electrically conductive coat 72 to the rod 40, high-frequency energy (current) can be supplied to the electrically conductive coat 72 via the rod 40. In this case, if high-frequency energy is applied to the treatment target that is clamped between the counter-surface 65 of the jaw-side electrode 64 and the electrically conductive coat 72, heat is generated in the treatment target by resistance heat generation, and the biological tissue is denatured.

In the case of the structure as illustrated in FIG. 5 in which electric power is supplied to the electrically conductive coat 72 via the rod 40, the wiring line 90 near the distal portion of the shaft 30 is needless. In this case, for example, within the housing 21, the rod 40 is electrically connected to the electric wire in the cable 110.

That part of the electrically conductive coat 72, which is disposed in the shaft 30, is connected to the rod 40. Thereby, high-frequency energy (current) flows to the treatment surface 76 of the electrically conductive coat 72 through the part of the electrically conductive coat 72, which is disposed in the shaft 30.

Thus, the temperature of the distal portion 41, which constitutes a part of the rod-side treatment section 70 in the rod 40, is prevented from rising to a high temperature. Note that, except for a contact part of the electrically conductive coat 72, which is put in contact with the rod 40 for the purpose of electric power supply, the electrically conductive coat 72 is not in contact with the rod 40. The electrically conductive coat 72, except for the above-described contact part, is disposed within the range of the heat-insulating coat 71.

In the modification illustrated in FIG. 5, too, the heat-insulating coat may be annually formed on the peripheral surface of the distal portion 41 of the rod 40, as illustrated in FIG. 4.

Additionally, the medical apparatus 10 can be constituted as a bipolar-type treatment instrument by way of example. As a modification, the medical apparatus 10 may be constituted as a monopolar-type treatment instrument, such as an electric scalpel, in which the end effector 50 is composed of only the rod 40.

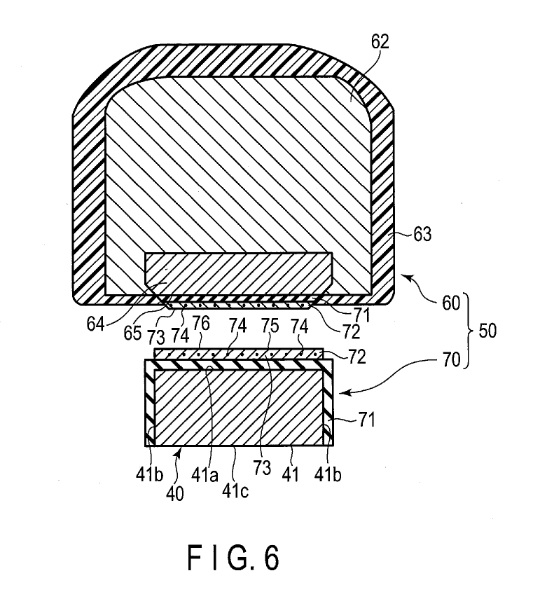

The heat-insulating coat 71 and electrically conductive coat 72 can be provided on the distal portion 41 of the rod 40. In another example, the heat-insulating coat 71 and electrically conductive coat 72 may be provided on the jaw 60. FIG. 6 is a cross-sectional view illustrating a modification relating to this example. As illustrated in FIG. 6, in this modification, a heat-insulating coat 71 is formed on the counter-surface 65 of the jaw-side electrode 64. An electrically conductive coat 72 is formed on this heat-insulating coat 71. In this modification, too, it is preferable that the electrically conductive coat 72 is formed within the range of the heat-insulating coat 71.

In the case of the configuration of this modification, the electrically conductive coat 72 provided on the jaw 60 is electrically connected to the jaw-side electrode 64. This electrical connection may be made such that a part of the electrically conductive coat 72 is directly connected to the jaw-side electrode 64. Alternatively, the electrical connection may be made by a wiring line. In the modification illustrated in FIG. 6, the conduction of heat to the jaw-side electrode 64 and jaw body 62 is made difficult.

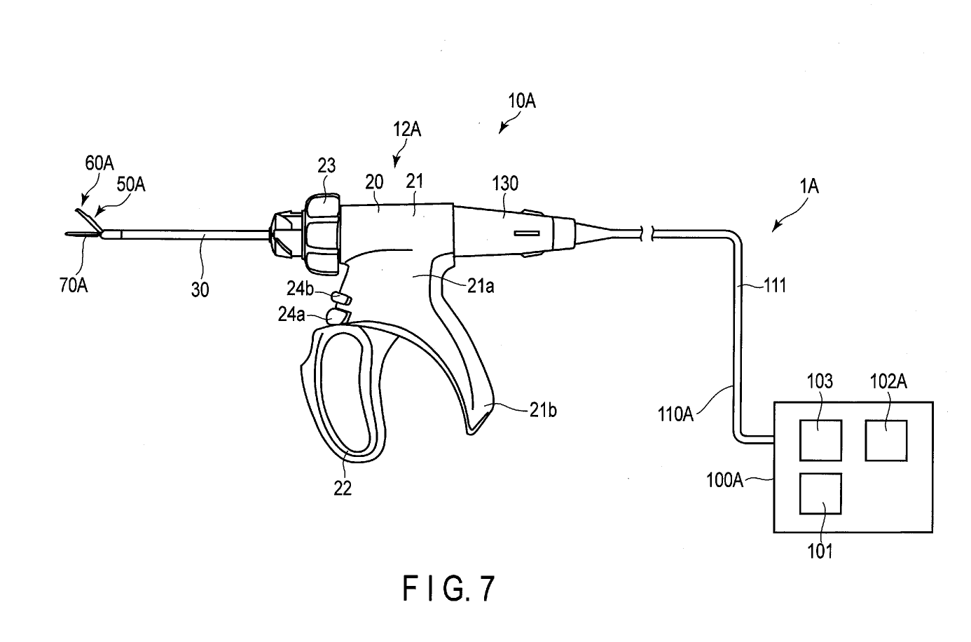

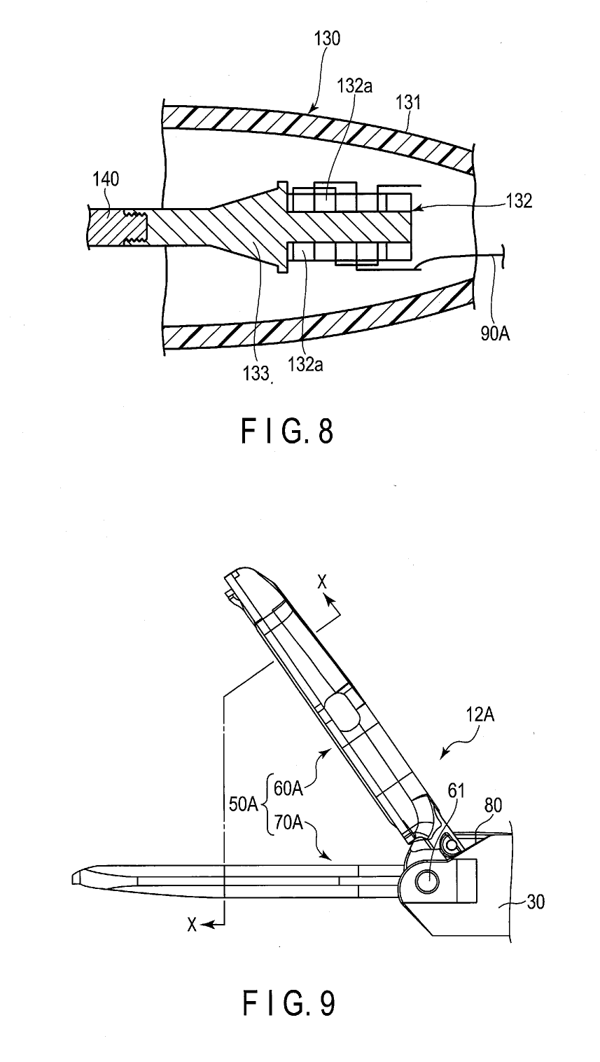

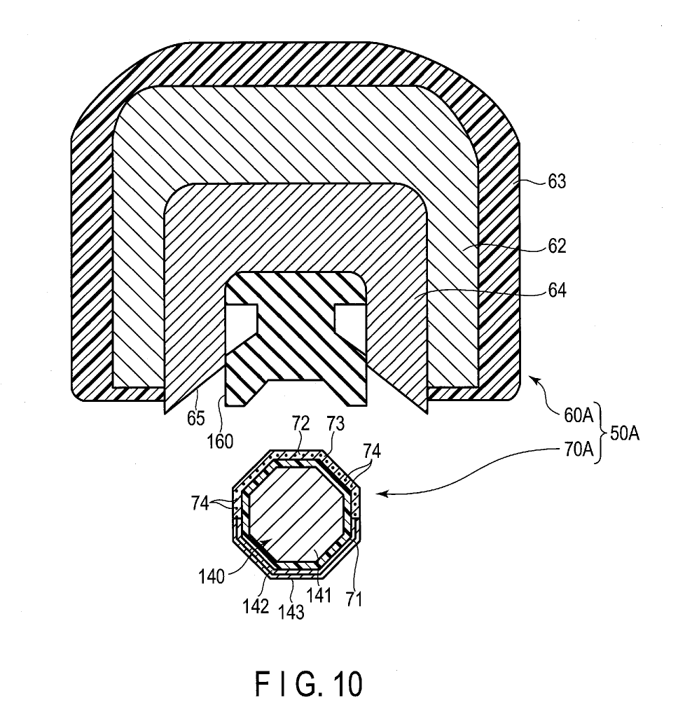

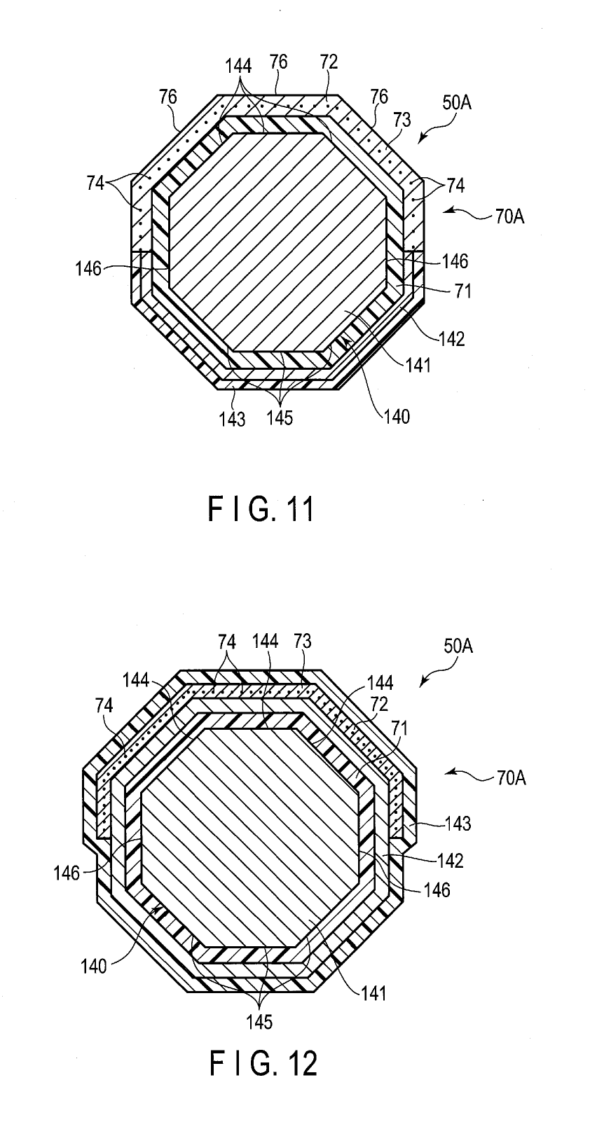

Next, a medical instrument of the present invention will be described with reference to FIG. 7 to FIG. 12, by taking a medical apparatus 10A as an example. FIG. 7 is a schematic view illustrating a medical system 1A including the medical apparatus 10A with a medical instrument 12A. FIG. 8 is a cross-sectional view illustrating a transducer 130 of the medical apparatus 10A. FIG. 9 is a side view illustrating an end effector 50A of the medical apparatus 10A. FIG. 10 is a cross-sectional view illustrating the end effector 50A. FIG. 11 is a cross-sectional view illustrating a rod-side treatment section 70A of the end effector 50A.

As illustrated in FIG. 7, the medical system 1A includes the medical apparatus 10A configured to treat a treatment target; an energy control device 100A configured to supply energy to the medical apparatus 10A; and a cable 110A connected to the medical apparatus 10A and energy control device 100A and configured to transmit energy from the energy control device 100A to the medical apparatus 10A.

In the present embodiment, for example, the medical apparatus 10A is configured to pass high-frequency energy through the treatment target, and configured to apply ultrasonic energy (ultrasonic vibration) to the treatment target.

The medical apparatus 10A includes a medical instrument 12A and a transducer 130 configured to be attachable/detachable to/from the medical instrument 12A. The medical instrument 12A includes a hand piece 20 a shaft 30 coupled to the hand piece 20 and partly disposed within the hand piece 20; a probe (rod) 140 disposed within the shaft 30 and hand piece 20; an effector 50A configured to clamp a treatment target by opening and closing, and configured to perform treatment on the treatment target; and a wiring line 90A configured to transmit energy to the transducer 130. The transducer 130 is configured to be attachable/detachable to/from the hand piece 20.

The hand piece 20 includes a first operation button 24a which is an example of an operation section provided on the housing 21; and a second operation button 24b which is an example of the operation section provided on the housing 21.

When the first operation button 24a is pressed, the end effector 50A is configured to treat the treatment target by high-frequency energy which is an example of energy.

When the second operation button 24b is pressed, the end effector 50A is configured to treat the treatment target by ultrasonic energy which is an example of energy.

FIG. 8 is a cross-sectional view illustrating the transducer 130. The transducer 130 is configured to generate ultrasonic vibration and is configured to transmit the ultrasonic vibration to the probe (rod) 140. As illustrated in FIG. 7 and FIG. 8, the transducer 130 includes a housing 131, an ultrasonic transducer 132 disposed in the housing 131, and a horn member 133 disposed in the housing 131.

The housing 131 is detachably attached to the housing body 21a. The housing 131 is disposed along an extension line of the axis of the probe 140 in the housing body 21a of the medical apparatus 10A.

The ultrasonic transducer 132 is attached to the horn member 133. The horn member 133 is formed of, for example, a metallic material. The horn member 133 includes a cross-section varying portion which has a substantially conical shape and has a cross section decreasing toward the distal side of the probe 140. The ultrasonic vibration generated by the ultrasonic transducer 132 is so-called longitudinal vibration, and the direction of vibration agrees with the longitudinal direction of the probe 140. In the cross-section varying portion of the horn member 133, the amplitude of ultrasonic vibration is increased.

The probe 140 is formed in a rod shape, like the rod 40 described previously. The probe 140 is formed such that ultrasonic vibration can be transmitted to a distal portion 141 of the probe 140. The probe (rod) 140 is used as a vibration transmitting member configured to treat the treatment target by ultrasonic vibration generated by the ultrasonic transducer 130. The probe 140 is formed of an electrically conductive material. That is, it is preferable that the probe (rod) 140 has an electric conductive property. The probe 140 has enough strength to perform treatment on the treatment target.

The probe 140 is disposed in the shaft 30, the housing 21 and the housing 131 of the transducer 130, in such a position that the axis of the probe 140 agrees with the axis of the shaft 30.

One end of the probe 140 is connected to a distal end of the horn member 133. The probe 140 is formed such that ultrasonic vibration can be transmitted from the horn member 133 to the probe 140. The other end of the probe 140 protrudes from the opening of the distal end of the shaft 30. Here, that portion of the probe 140, which protrudes from the opening of the distal end of the shaft 30, is defined as the distal portion 141.

As illustrated in FIG. 9, the end effector 50A includes a jaw 60A rotatably provided on the distal portion of the shaft 30, and a rod-side treatment section 70A provided on the distal portion 141 of the probe 140. The rotational structure of the jaw 60A relative to the shaft 30 is the same as the rotational structure of the jaw 60 relative to the shaft 30, which was described previously.

As illustrated in FIG. 10, the jaw 60A includes a jaw body 62, a cover 63, a jaw-side electrode 64, and a pad 160 fixed to the jaw-side electrode 64.

The jaw-side electrode 64 includes a counter-surface 65 opposed to the probe 140 in the opening/closing direction. The counter-surface 65 of the jaw-side electrode 64 is recessed. The pad 160 is disposed in the recess of the counter-surface 65. The pad 160 is fixed to the jaw-side electrode 64. The pad 160 is formed of, for example, a resin material having an electrical insulation property, heat resistance and wear resistance, such as PTFE material.

FIG. 11 is a cross-sectional view illustrating, in enlarged scale, the treatment section 70A. FIG. 11 illustrates a state in which the treatment section 70A is cut along a cross section perpendicular to the axis of the distal portion 141 of the probe 140. As illustrated in FIG. 11, the treatment section 70A includes the distal portion (base member) 141 of the probe 140, which protrudes outward from the distal end of the shaft 30; a heat-insulating coat 71 formed on the distal portion 141; an electrically conductive coat 72 formed on the heat-insulating coat 71; a reinforcement coat 142 formed on the heat-insulating coat 71; and a water-repellent coat 143 formed on the reinforcement coat 142.

The distal portion 141 of the probe 140 is disposed in a position opposed to the pad 160 in the opening/closing direction of the jaw 60A. In the state in which the jaw 60A is closed against the distal portion 141, the distal end of the distal portion 141 is located at substantially the same position as the distal end of the jaw 60A.

FIG. 11 is a cross-sectional view which illustrates, in enlarged scale, the distal portion 141 of the probe 140. As illustrated in FIG. 11, the distal portion 141 of the probe 140 has, for example, an octagonal cross section which is perpendicular to the axis of the distal portion 141.

Three surfaces on the jaw 60A side, among peripheral surfaces of the distal portion 141, constitute a counter-surface 144 which is opposed to the jaw-side electrode 64 and pad 160. Three surfaces on the opposite side to the counter-surface 144, among the peripheral surfaces of the distal portion 141, constitute an opposite surface 145. A pair of side surfaces 146 are disposed between the counter-surface 144 and the opposite surface 145.

In the present embodiment, the heat-insulating coat 71 is formed on the peripheral surfaces of the distal portion 141. The peripheral surfaces, in this context, are surfaces around the axis of the distal portion 141, and are the counter-surface 144, opposite surface 145 and the pair of side surfaces 146. The heat-insulating coat 71 is formed in an annular shape. The heat-insulating coat 71 has a uniform, or substantially uniform, thickness. Thus, a shape formed by an outer surface in the cross section of the heat-insulating coat 71 is an octagonal shape.

In the present embodiment, the heat-insulating coat 71 is formed so as to make it possible for the heat of the electrically conductive coat 72 to be less easily conducted to the distal portion 141 of the probe 140. In the present embodiment, the specific heat capacity of the heat-insulating coat 71 is greater than that of the probe 140. The thermal conductivity of the heat-insulating coat 71 is lower than that of the probe 140.

The electrically conductive coat 72 is formed on that part of the heat-insulating coat 71, which is opposed to the pad 160 and jaw-side electrode 64. The electrically conductive coat 72 is formed on the heat-insulating coat 71 which is formed on the counter-surface 144 of the distal portion 141. The electrically conductive coat 72 is formed on the heat-insulating coat 71 that is formed on those parts of the side surfaces 146 of the distal portion 141, which are located on the jaw 60A side. The surface of the electrically conductive coat 72 can come in contact with the treatment target, and serves as a treatment surface 76.

In the present embodiment, for example, the electrically conductive coat 72 is formed such that high-frequency energy (current) can be supplied from the energy control device 100A to the electrically conductive coat 72 via the probe 140. Electric power is supplied to the electrically conductive coat 72 via the probe 140 by the same structure as described in FIG. 5, in which electric power is supplied to the electrically conductive coat 72 via the rod 40.

Concretely, the high-frequency energy supply 101 is electrically connected to the probe 140. Thus, electric energy is supplied from the high-frequency energy supply 101 to the probe 140. A part of the electrically conductive coat 72 is electrically connected to the probe 140. As this electrical connection, the electrically conductive coat 72 may be directly connected to the probe 140. By the electrical connection of the electrically conductive coat 72 to the probe 140, high-frequency energy can be supplied to the electrically conductive coat 72 via the probe 140. It is preferable that the electrically conductive coat 72 is connected to a portion of the probe 140, which is other than the distal portion 141 that constitutes a part of the treatment section 70A. It is preferable that the electrically conductive coat 72 is connected to the probe 140 at a position away from the distal portion 141 of the probe 140.

Except for a contact part of the electrically conductive coat 72, which is put in contact with the probe 140 for the purpose of electric power supply, the electrically conductive coat 72 is not in contact with the probe 140. Thus, the electrically conductive coat 72, except for the above-described contact part, is disposed within the range of the heat-insulating coat 71.

The heat capacity of the electrically conductive coat 72 of can be less than the heat capacity of the distal portion 141 of the probe 140. The thermal conductivity of the electrically conductive coat 72 of the present embodiment can be lower than the thermal conductivity of the probe 140. Thus, in the present embodiment, too, since the electrically conductive coat 72 has a high heat radiation property than the distal portion 141, the heat of the electrically conductive coat 72 is not easily conducted to the distal portion 141.

The reinforcement coat 142 is formed such that the reinforcement coat 142 can improve the strength of the treatment section 70A. The reinforcement coat 142 is formed on a part of the heat-insulating coat 71, which is opposed to the opposite surface 145 of the distal portion 141, and on parts of the side surfaces 146. Note that the parts of the side surfaces 146, on which the reinforcement coat 142 is formed, are parts on which the electrically conductive coat 72 is not formed.

It is preferable that the reinforcement coat 142 is formed to have a uniform, or substantially uniform, thickness. The reinforcement coat 142 is formed to have a proper thickness in a range of, e.g. several μm to several-hundred μm, in accordance with an internal organ, an organ or a tissue, which is a treatment target. Although the reinforcement coat 142 is formed of a resin material such as PEEK, the reinforcement coat 142 may be formed of other resins, if such resins have electrical insulation properties, and proper heat resistance and wear resistance.

The water-repellent coat 143 is formed on the reinforcement coat 142. The water-repellent coat 143 has water repellency. The water-repellent coat 143 has a uniform, or substantially uniform, thickness. The total thickness of the thickness of the water-repellent coat 143 and the thickness of the reinforcement coat 142 is determined so as to become equal or substantially equal to the thickness of the electrically conductive coat 72. Thus, the cross-sectional shape of the treatment section 70A is octagonal, as illustrated in FIG. 11.

The end effector 50A with this structure can clamp a biological tissue between the jaw 60A and the rod-side treatment section 70A, by the opening/closing of the jaw 60A relative to the rod-side treatment section 70A.

The energy control device 100A includes an ultrasonic energy supply 103, a high-frequency energy supply 101, and a controller 102A which configured to control the ultrasonic energy supply 103 and high-frequency energy supply 101.

The ultrasonic energy supply 103 is configured to supply electric energy which is suited to the generation of ultrasonic in the transducer 130. The high-frequency energy supply 101 is configured to supply, to the probe 140, electric energy which enables a proper treatment in the end effector 50A.

The controller 102A is configured to supply electric energy from the high-frequency energy supply 101, when the first operation button 24a is operated by the surgeon. The controller 102A is configured to supply electric energy from the ultrasonic energy supply 103 to the ultrasonic transducer 132 of the transducer 130, when the second operation button 24b is operated by the surgeon.

The wiring line 90A is electrically connected to the ultrasonic transducer 132 of the transducer 130.

The cable 110A includes a sheath 111, and an electric wire disposed in the sheath 111. The electric wire is electrically connected to the wiring line 90A, and is configured to supply electric power from the ultrasonic energy supply 103 to the wiring line 90A. Besides, electric power is supplied to the probe 140 by another electric wire.

Next, the function of the medical apparatus 10A will be described.

In a treatment, the surgeon can clamp the treatment target by the end effector 50A by operating the handle 22. In the state in which the treatment target is clamped by the end effector 50, the surgeon operates the first operation button 24a. Thereby, high-frequency energy can be applied to the treatment target clamped by the end effector 50A.

If the high-frequency energy is applied to the biological tissue in the treatment target, heat is generated in the biological tissue by resistance heat generation, and the biological tissue is denatured. By utilizing the denaturing, a treatment such as coagulation, or coagulation/cutting is performed to the biological tissue in the treatment target.

If the second operation button 24b is operated by the surgeon, the controller 102A causes the ultrasonic transducer 132 to generate vibration, and ultrasonic energy is applied to the treatment target via the probe 140. By the ultrasonic energy, a treatment of coagulation/cutting or only coagulation is performed on the biological tissue.

Besides, the temperature of the electrically conductive coat 72 is raised by the heat generated by the electrical resistance of the electrically conductive coat 72, and by the heat transferred from the treatment target. However, the electrically conductive coat 72 is formed on that surface of the heat-insulating coat 71, which is opposed to the jaw 60A in the opening/closing direction of the jaw 60A. The heat-insulating coat 71, which makes difficult the transfer of heat, is formed between the electrically conductive coat 72 and the probe 140. Thus, heat is not easily conducted from the electrically conductive coat 72 to the probe 140.

In the medical apparatus 10A of the present embodiment, the electrically conductive coat 72 is formed on the counter-surface of the heat-insulating coat 71, which is opposed to the jaw 60A in the opening/closing direction of the jaw 60A, and the reinforcement coat 142 is formed on the surface of the heat-insulating coat 71, which is opposite to the counter-surface of the heat-insulating coat 71. The water-repellent coat 143 is formed on the reinforcement coat 142.

However, the formation of the electrically conductive coat 72 is not limited to the case in which the electrically conductive coat 72 is formed on the surface of the heat-insulating coat 71. Another layer may be interposed between the electrically conductive coat 72 and the heat-insulating coat 71. In short, the heat-insulating coat 71 may be provided between the electrically conductive coat 72 and the probe 140, so as to make it possible for the heat of the electrically conductive coat 72 to be less easily conducted to the distal portion 141 of the probe 140. It should suffice if the electrically conductive coat 72 is provided in an outer layer (upper layer) with respect to the heat-insulating coat 71.

FIG. 12 is a cross-sectional view illustrating a modification of the medical apparatus 10A of the present embodiment. FIG. 12 illustrates a state in which the medical apparatus 10A is cut along a cross section perpendicular to the axis of the distal portion 141. FIG. 12 illustrates an example in which another layer is formed between the electrically conductive coat 72 and the heat-insulating coat 71, as described above.

As illustrated in FIG. 12, the reinforcement coat 142 may be formed on the entire surface of the heat-insulating coat 71. The electrically conductive coat 72 may be formed on the counter-surface of the reinforcement coat 142, which is opposed to the jaw 60A in the opening/closing direction of the jaw 60A.

In this manner, even when the electrically conductive coat 72 is formed on the reinforcement coat 142, it is preferable that the electrically conductive coat 72 is formed in the inside of the range of the heat-insulating coat 71.

As illustrated in FIG. 12, the water-repellent coat 143 may be formed on the electrically conductive coat 72 and reinforcement coat 142. Although the water-repellent coat 143 is insulative, the thickness of the water-repellent coat 143 is small. Thus, there is no hindrance in use when high-frequency energy is passed from the electrically conductive coat 72 to the jaw body 62, and there is no problem with the function of the medical apparatus 10A.

In the present embodiment, the example was described in which the probe 140 is used for supplying electric power to the electrically conductive coat 72, but the embodiment is not limited to this. For example, a wiring line may be electrically connected to the electrically conductive coat 72, and electric power may be supplied to the electrically conductive coat 72 by using this wiring line.

In this case, the heat-insulating coat 71 electrically insulates the electrically conductive coat 72 from the probe 140. In other words, the electrically conductive coat 72 is prevented from coming in contact with the probe 140 by the heat-insulating coat 71.

Additionally, in the present embodiment, the medical apparatus 10A is configured such that high-frequency energy is passed through the treatment target by the operation of the first operation button 24a. Ultrasonic energy is applied to the treatment target by the operation of the second operation button 24b. However, the embodiment is not limited to this. In one example, the medical apparatus 10A may be configured such that high-frequency energy and ultrasonic energy can be input to the treatment target by the operation of one operation button. For example, such a configuration may be adopted that electric power is supplied to the electrically conductive coat 72 and ultrasonic transducer 132 by operating the second operation button 24b.

Besides, in the second embodiment, the heat-insulating coat 71 and electrically conductive coat 72 are provided on the distal portion 141 of the probe 140. In another example, the heat-insulating coat 71 and electrically conductive coat 72 may be provided on the jaw 60A.

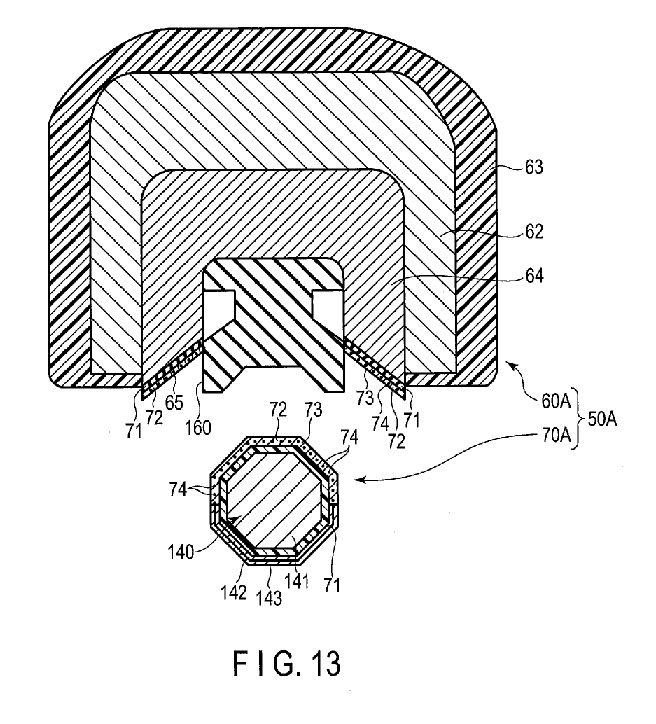

FIG. 13 is a cross-sectional view illustrating a modification relating to this example. As illustrated in FIG. 13, in this modification, the heat-insulating coat 71 is formed on the counter-surface 65 of the jaw-side electrode 64. The electrically conductive coat 72 is formed on the heat-insulating coat 71. In this modification, too, it is preferable that the electrically conductive coat 72 is formed within the range of the heat-insulating coat 71.

In the case of the configuration of this modification, the electrically conductive coat 72 provided on the jaw 60A is electrically connected to the jaw-side electrode 64. This electrical connection may be made such that a part of the electrically conductive coat 72 is directly connected to the jaw-side electrode 64. Alternatively, the electrical connection may be made by a wiring line. In the modification illustrated in FIG. 13, the conduction of heat to the jaw-side electrode 64 and jaw body 62 is made difficult.

Additionally, silver particles can be used in the electrically conductive coat 72. In another example, the particles may be another kind of metallic particles, which are different from the silver particles, such as copper particles with good electrical conductivity.

The medical apparatus 10 and 10A were described as examples of the medical instrument. However, the medical instrument is not limited to the medical apparatus 10 and medical apparatus 10A. In another example, the medical instrument can be used as a medical instrument which supplies electric energy in a state of direct contact with a biological tissue. Note that for the medical apparatus 10 or the medical apparatus 10A, the rigid member can have such strength as to be capable of treating the treatment target.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical apparatus comprising:
a rod that includes two side surfaces;
a heat-insulating coat having a heat insulating property;
the heat-insulating coat formed on a part of the rod including the two side surfaces of the rod;
the heat-insulating coat configured to suppress conduction of heat from a treatment target to the rod; and
an electrically conductive coat having an electric conductive property;
the electrically conductive coat being formed on the heat-insulating coat and positioned such that the electrically conductive coat is configured to contact the treatment target.

2. The medical apparatus of claim 1, wherein:
a specific heat capacity the heat-insulating coat is greater than a specific heat capacity of the rod, and
a thermal conductivity of the heat-insulating coat is lower than a thermal conductivity of the rod.

3. The medical apparatus of claim 2, wherein the heat-insulating coat is formed of one of: a ceramic material and a heat-resistant material.

4. The medical apparatus of claim 1, wherein:
a heat capacity of the electrically conductive coat is less than a heat capacity of the rod, and
a thermal conductivity of the electrically conductive coat is lower than a heat capacity of the rod.

5. The medical apparatus of claim 4, wherein the electrically conductive coat includes:
an electrically non-conductive coat being configured to repel water; and
a plurality of electrically conductive particles dispersed in the electrically non-conductive coat.

6. The medical apparatus of claim 5, wherein the electrically non-conductive coat includes a fluororesin.

7. The medical apparatus of claim 5, wherein the electrically conductive particles are silver particles.

8. The medical apparatus of claim 1, wherein:
the rod has an electric conductive property, and
the electrically conductive coat is electrically insulated from the rod by the heat-insulating coat.

9. The medical apparatus of claim 1, comprising a transducer configured to generate ultrasonic vibration and configured to transmit the ultrasonic vibration to the rod.

10. The medical apparatus of claim 1, comprising a jaw configured to clamp the treatment target between the jaw and the electrically conductive coat,
wherein the jaw includes a jaw-side electrode configured to pass the high-frequency energy between the jaw-side electrode and the electrically conductive coat.

11. The medical apparatus of claim 10, wherein the electrically conductive coat is opposed to the jaw-side electrode.

12. The medical apparatus of claim 11, wherein the electrically conductive coat is used as an electrode configured to pass the high-frequency energy through the treatment target clamped between the electrically conductive coat and the jaw-side electrode.

13. The medical apparatus of claim 1, wherein the rod is used as a vibration transmitting member configured to treat the treatment target by ultrasonic vibration generated by an ultrasonic transducer.

14. The medical apparatus of claim 13, comprising a reinforcement coat having wear resistance,
wherein the reinforcement coat is formed on the heat-insulating coat.

15. The medical apparatus of claim 14, comprising a water-repellent coat configured to repel water, the water-repellent coat being formed on the reinforcement coat.

16. The medical apparatus of claim 1, wherein the electrically conductive coat is configured to treat the treatment target with high-frequency energy.
